(19) United States
(12) Patent Application Publication
(10) Pub. No.: US 2015/0238729 A1
(43) Pub. Date: Aug. 27, 2015

(54) Cardiac Access Catheter, System, and Method

(71) Applicants: Mark Lynn Jenson, Greenfield, MN (US); William Joseph Drasler, Minnetonka, MN (US); Richard C. Kravik, Champlin, MN (US); William Joseph Drasler, II, Minnetonka, MN (US)

(72) Inventors: Mark Lynn Jenson, Greenfield, MN (US); William Joseph Drasler, Minnetonka, MN (US); Richard C. Kravik, Champlin, MN (US); William Joseph Drasler, II, Minnetonka, MN (US)

(21) Appl. No.: 14/628,495

(22) Filed: Feb. 23, 2015

Related U.S. Application Data
(60) Provisional application No. 61/966,478, filed on Feb. 24, 2014.

(51) Int. Cl. A61M 25/01; A61M 25/09; A61M 25/00

(52) U.S. Cl. CPC A61M 25/0102; A61M 25/0043; A61M 25/09

(57) ABSTRACT

A system for accessing a desired location in the heart or other target areas has an alignment catheter which directs a directional element towards a target area. A second directional element can be directed towards a target tissue, using the known location of the first directional element as a basis to easily locate other tissues and targets. One or more stabilization elements can help to stabilize the distal portion of the apparatus near the target area. The system can be used for transvenous access to the transatrial septum for transatrial access to the mitral valve, for example, and can use a stabilization basket near the right atrium. The system can be used for coronary sinus access or access to other targets. Various configurations are disclosed, as are various stabilization elements, directional elements, combinations, and methods.

### RELATED PATENT DOCUMENTS

This patent application claims benefit from the earlier filed U.S. Provisional Application No. 61/966,478 filed Feb. 24, 2014 entitled “Cardiac Access Catheter, System, and Method”, pending, which is hereby incorporated into this application by reference as if fully set forth herein.

### BACKGROUND

1. Field of the Invention

The present invention pertains generally to medical devices, and particularly to medical devices for catheter-based treatments, and more particularly, for intravascular access to portions of the heart and associated vasculature.

2. Description of the Prior Art

Access to specific areas of the heart and associated vasculature is required for various catheter-based diagnostic and therapeutic procedures. In some situations, there is a need to intravascularly access the coronary sinus and associated structures such as the great cardiac vein and its branches. In other situations, there is a need to intravascularly access the left atrium, such as via trans-septal access (passing through the inter-atrial septum from the right side into the left atrium) or through a patent foramen ovale (PFO), and so forth. Some of the situations where such access is needed, include access for placement of devices to treat an improperly functioning mitral valve. Other situations include access for placement of electrophysiology devices such as electrodes, leads, and associated equipment for pacing, sensing, or cardioversion, or defibrillation.

Still other situations include access to address issues with electrophysiology, septal defects, atrial appendage, other cardiac valves, cardiac wall pathologies, coronary artery pathologies, and so forth. Diagnostic and therapeutic procedures, including placement of implants, access for local drug delivery, and other procedures, may require safe and reliable access to specific tissues or areas in and near the heart. In some situations, less-invasive approaches are preferred, such as via remote vascular access via femoral artery, femoral vein, subclavian vein, or other vascular approach, or smaller-incision approaches such as mini-thoracotomy, or trans-apical approaches.

Various devices and methods are used to access various structures in and near the heart for such diagnostic and therapeutic procedures. Navigating to access the desired anatomical locations can be challenging, however. Various guidewires, catheters, and imaging tools have been introduced to address these challenges, but access can still be difficult or impossible, or can require expensive or complicated equipment and procedures. Vascular complications and other safety problems can occur, and access can be time-consuming.

Puncturing the interatrial septum in the wrong location can cause safety and effectiveness problems. Incorrect puncture can cause hemorrhagic complications, with bleeding from the atrium into the extra-cardiac tissues and potential spaces, or could puncture the aorta, the esophagus, or other important tissues. Important cardiac electrophysiological structures, and coronary vasculature, are also nearby. Even if the puncture is safe, positioning the puncture at a location that is in the wrong location or orientation can make it difficult or impossible to properly position a therapeutic device within the left atrium. For example, trans-septal access originating from the inferior vena cava that is too far cranial, can make it difficult to guide a therapy device to the mitral valve and maintain any required alignment with the axis of the mitral valve.

There is continued need for improved devices and methods for safe, reliable, and efficient access to various cardiac structures for diagnostic and therapeutic procedures. There is particular need for improved devices and methods for accessing the coronary sinus, and devices and methods for trans-septal access to the left atrium. Among other utility, such devices and methods can facilitate placement of devices in the coronary sinus and great cardiac vein, and placement of devices at the mitral valve.

### SUMMARY OF THE INVENTION

The general purpose of the present invention is to provide a better way of accessing left heart structures for diagnostic and therapeutic procedures.

According to embodiments of the present invention, there is provided apparatus for trans-septal access to the mitral valve.

According to embodiments of the present invention, there is provided apparatus for transvenous access to the coronary sinus.

According to embodiments of the present invention, there is provided apparatus for trans-septal access to the left atrium.

According to embodiments of the present invention, there is provided apparatus for transvenous access to the interatrial septum.

According to embodiments of the present invention, there are provided methods for trans-septal access to the mitral valve.

According to embodiments of the present invention, there are provided methods for transvenous access to the coronary sinus.

According to embodiments of the present invention, there are provided methods for trans-septal access to the left atrium.

According to embodiments of the present invention, there are provided methods for transvenous access to the interatrial septum.

According to embodiments of the present invention, there are provided apparatus and methods for positioning and navigating devices within a vessel or vascular system.

In one example, the present invention comprises a system for accessing a desired location in the heart from a transvenous route. For example, a percutaneous femoral vein access can be used, a catheter of the present invention is advanced cranially via the inferior vena cava and positioned so that a stabilizing element is secured at or near the right atrium. Alignment elements direct a directional element towards a desired location such as the coronary sinus ostium. The present invention may include alignment adjustment features which provide controlled movement of the directional element with respect to the stabilizing element, to provide for directing the directional element towards a desired axial distance and rotational orientation with respect to the stabilizing element. The stabilizing element can be structured with an asymmetric shape such that the axis of the catheter or apparatus is not aligned with the axis of the stabilizing member; the catheter can thereby be held by the basket into a position adjacent one wall of the vascular structure for improved advancement of the directional catheter across the vascular structure with better force transmission and location.

The various elements of the apparatus can take various forms as are described more fully in the detailed description to follow, but as an example apparatus, method, and use, a system including an outer catheter with a distal basket is advanced to a point at or near the right atrium, and the basket is expanded to contact vascular structures to stabilize the distal portion of the outer catheter. A directional catheter is advanced form the distal portion of the system, towards the coronary sinus ostium. The orientation of the directional catheter can be adjusted, such as by rotating elements near the distal end of the system without contracting the stabilizing basket, or by temporarily contracting the stabilizing basket and rotating the outer catheter as needed, according to imaging or other guidance. Once the orientation of the directional catheter is as desired, the directional catheter is advanced in the direction desired, to access the coronary sinus ostium. After the tip of the directional catheter is in, at, or near the coronary sinus ostium, the coronary sinus ostium is accessed by further advancement of the directional catheter, or by advancing a guidewire out from the distal portion of the directional catheter. Such coronary sinus access can be useful, for example, for placing pacing or other electrodes, or for placing a mitral annuloplasty device.

In another example, the present invention comprises a system for accessing a desired location in the heart from a transvenous route. In this example, however, a subclavian vein access is used, and a catheter of the present invention is advanced caudally via the superior vena cava and positioned so that a stabilizing element is secured at or near the right atrium. In yet other examples, a jugular vein access is used.

In some embodiments more than one stabilizing element can be used. For example, two stabilizing baskets can be used, positioned a short distance apart, so that the distal portion of the directional element can be located between the two stabilizing baskets. This can be particularly useful, for example, when a suitable stabilizing location in the vasculature is a distance from the desired location of the directional element, whereby stabilization both proximal and distal to the directional element can improve the stabilization of the distal portion of the apparatus. Other stabilizing elements can be used instead of or in addition to baskets. For example, braided structures similar to certain intravascular stents can be used, one or more rings, axial struts, or spiral structures can be used, an inflatable balloon can be used, and so forth.

Preferably, the structure allows blood flow past the stabilizing elements, although in some situations, such as applications that are not within a blood vessel, this is not required. For example, navigating within the urinary system, or a cerebrospinal fluid space, or within the trachea or bronchial tree, or within the gastrointestinal system, or extravascular access path, can benefit from the stabilization and directional control obtained with the present invention.

One particularly advantageous example of the present invention is a system for trans-septal access (from a percutaneous venous access, across the interatrial septum) for access to the left atrium for mitral valve repair or replacement. In this case, similar stabilization element(s) are used to direct a directional element to a safe location in the interatrial septum, where a piercing device can be used to puncture an access hole in the septum. In this case, the directional element can perform the septal puncture, or the directional element can be used to guide a piercing device to the desired location in the septum.

In other examples, the present invention provides apparatus and methods for access to the left atrial appendage for closure of the appendage, or access to an atrial septal defect for repair or closure of the defect, or access to a patent foramen ovale to close the patent foramen ovale, or access to specific locations within the left atrium for electrophysiology measurements or for treatment of atrial fibrillation. In some examples, the desired location can be within the right atrium, for electrophysiology measurements or therapies.

In yet another example, the apparatus provides for at least a first directional element and a second directional element. In this example, the first directional element can be directed to a first specific location, such as the coronary sinus, and temporarily left in place in that location. With the stabilizing element(s) and the first directional element in place, the orientation of the distal end of the apparatus is stabilized and fixed relative to the known location of the first specific location. Then, the second directional element is directed to a second specific location, such as the fossa ovalis. If the relative locations of the first and second specific locations are known to be typically a certain orientation (with respect to rotational angle around the axis of the alignment catheter or apparatus) and distance from each other, locating the second specific location is facilitated by this apparatus and method. For example, if the fossa ovalis is typically in the area of a certain range of distance cranial to the coronary sinus ostium, and a certain distance anterior, posterior, medial, and/or lateral to the coronary sinus ostium, and oriented typically in a certain angular orientation, then stabilizing and fixing the apparatus with respect to the first specific location (such as the coronary sinus ostium) can greatly simplify locating of the second specific location. Then, a small amount of “searching” by relative movements of components, tactile feedback, contrast injection or other imaging, and so forth, as are known skills of interventionalists, can successfully locate the second specific location with greater ease, confidence, and safety, than without the use of the present invention. In many cases, the alignment and location can be determined sufficiently well by the present invention, that no “searching” at all is required to locate the second specific location and place a directional element at that desired location.

The present invention, by providing safer and more reliable access to cardiac tissues and locations, may reduce the dependency on certain imaging and guiding systems. For example, optimal performance of certain access procedures presently may require both fluoroscopic and echocardiographic imaging; utilizing both can increase the cost and complexity of the procedure.

Therefore, utilizing the present invention may be of significant benefit in reducing the cost and complexity of the procedure, and allow access to cardiac tissues and locations with less imaging system requirements.

One significant aspect and feature of the present invention is an access system with a stabilizing element which helps maintain the system in a consistent location and orientation within a body vessel or cavity.

Another significant aspect and feature of the present invention is an access system with a directional element which can be located and oriented within a body vessel or cavity by relationship with the stabilizing element and thereby by relationship with the body vessel or cavity, to direct the directional element towards a desired location in the body.

Yet another significant aspect and feature of the present invention is an access system with a directional element and a stabilizing element, where the directional element is adjustable and locatable with respect to the stabilizing element, to allow the directional element to be carefully located directed while the stabilizing element maintains a stable position and orientation in the body vessel or cavity.

Another significant aspect and feature is locating a first directional element into a known body orifice or cavity and then using this first location to enable location of a second directional element into a second body orifice or cavity at a specified distance and angle with respect to the first directional element.

Having thus briefly described one or more embodiments of the present invention, and having mentioned some significant aspects and features of the present invention, it is the principal object of the present invention to provide apparatus and methods facilitating access to a location within the body to facilitate a diagnostic or therapeutic procedure.

A further object of the present invention is to provide safe access to locations within or near the heart. Additional objects of embodiments of the present invention include: providing access from an inferior vein such as the femoral vein, providing access from a superior vein such as the subclavian vein or the jugular vein, providing access to the coronary sinus, providing access to the fossa ovalis, providing access to portions of the right atrium or interatrial septum, providing access to the right ventricle, providing access to a cardiac valve, providing access to a device within or near the heart, providing trans-septal access to portions of the left atrium, providing access to the pulmonary veins, providing access to the mitral valve, providing access to the left ventricle, providing access to the aortic valve, providing access to one or more great vessels, providing access to a patent ductus arteriosis, providing access via percutaneous or direct arterial access, and/or providing access via laparoscopic or other endoscopic or less-invasive approach.

A further object of the present invention is to assist in the placement of a transcatheter mitral valve replacement (TMVR) device or mitral valve repair device via a catheter from the superior or inferior vena cava that crosses the interatrial septum. Placement of a TMVR device across the mitral annulus requires that the delivery catheter is directed along the same axis as the mitral annulus so that the TMVR device is aligned with the mitral axis and is not cocked. Crossing the septum can be difficult even when using transesophageal echo (TEE) or intracardiac echo (ICE). Entrance from the inferior vena cava to cross the septum is preferred by some physicians because this approach places the catheter in alignment with the foramen ovale for passage into the left atrium. Also, the catheter will often fall tactilely into the fossa ovalis for puncture across the septum. One difficulty with this approach is that the catheter must make a significant turn and form a loop in the left atrium before it can be directed across the mitral annulus. A loop in a stiff TMVR catheter can result in the catheter becoming misaligned with the axis of the mitral valve; the result is the potential misalignment of the TMVR with the mitral annulus axis.

The present apparatus allows an improved delivery of a stiff and large TMVR catheter across the septum via an access from the superior vena cava. Such an access across the septum from a superior approach is currently not a preferred choice for the physician. However with the apparatus of the present invention, this superior approach is both reliable and allows improved alignment with the axis of the mitral annulus. The present invention first provides access to a first orifice that is more easily accessed from the superior approach such as the coronary sinus ostium. The position of the interatrial septum that is suitable for puncture and access from the right to the left atrium is positioned at a specific distance cranial from the coronary sinus. The septal puncture can be created at a specific angle that is generally at the same or similar angle as that used for access to the coronary sinus and with respect to the axis of the catheter of the present apparatus. The septal puncture made from the superior approach provides a more direct angle of approach to the mitral annulus without requiring the TMVR catheter to undergo significant bending. The TMVR catheter can then be in an improved alignment with the axis of the mitral valve. The septal puncture can be made without the potential for accidental puncture to neighboring vessels or cavities that could result in patient bleeding due to improper puncture of a neighboring vessel or death.

### BRIEF DESCRIPTION OF THE DRAWINGS

Other objects of the present invention and many of the attendant advantages of the present invention will be readily appreciated as the same becomes better understood by reference to the following detailed description when considered in connection with the accompanying drawings, in which like reference numerals designate like parts throughout the figures thereof and wherein:

FIG. 1A illustrates a cardiac access system of the present invention.

FIG. 1B illustrates an alternative cardiac access system of the present invention.

FIG. 2A further illustrates the cardiac access system of FIG. 1A, and methods of access to the coronary sinus ostium from an inferior transvenous access.

FIG. 2B further illustrates the cardiac access system of FIG. 1A, and methods of access to the coronary sinus ostium from a superior transvenous access.

FIG. 3A further illustrates the cardiac access system of FIG. 1B, and illustrates methods of access to the coronary sinus ostium and the fossa ovalis, from an inferior transvenous access.

FIG. 3B further illustrates the cardiac access system of FIG. 1B, and illustrates methods of access to the coronary sinus ostium and the fossa ovalis, from a superior transvenous access.

FIG. 4A illustrates an alternative cardiac access system and methods of superior transvenous access to the transatrial septum.

FIG. 4B illustrates an axial view of the system of FIG. 4A.

FIG. 5A illustrates example stabilization means comprising an expandable coil.

FIG. 5B illustrates example stabilization means comprising a helical balloon.

FIG. 5C illustrates example stabilization means comprising a dual opposed balloons.

FIG. 5D illustrates example stabilization means comprising a staggered opposed balloons.

FIG. 5E illustrates example stabilization means comprising an expandable basket constrained at both ends.

FIG. 5F example stabilization means comprising an expandable basket constrained at one end.

FIG. 5G illustrates example stabilization means comprising an alternative expandable basket constrained at one end.

### DETAILED DESCRIPTION OF THE PREFERRED EMBODIMENTS

It is desired to direct a device to specific tissues and locations within the body to facilitate diagnosis, monitoring, treatment, or excision procedures in the body. While access to some tissues and locations is relatively simple, certain locations are difficult to access, with sufficient safety, ease, and reliability. This is especially true when it is desired to approach the tissues or locations in a less-invasive or minimally-invasive manner, as opposed to a more-invasive conventional surgical approach. For example, endoscopic, or catheter-based diagnosis and therapy, including percutaneous vascular approaches, are preferred when they can be performed safely and effectively.

It is particularly desirable to access portions of the heart and nearby structures from a percutaneous approach via a remote blood vessel. For example, it is desired to access left heart structures via transvenous routes, such as an inferior route from a femoral venous access, for example, or a superior route from a subclavian venous access, for example. However, navigating a catheter-based device within the vasculature and through the heart to reach the desired location can be difficult, and can have safety and effectiveness problems. These problems increase mortality, morbidity, and cost, including special precautions that may be undertaken to minimize the likelihood or consequences of these problems or complications.

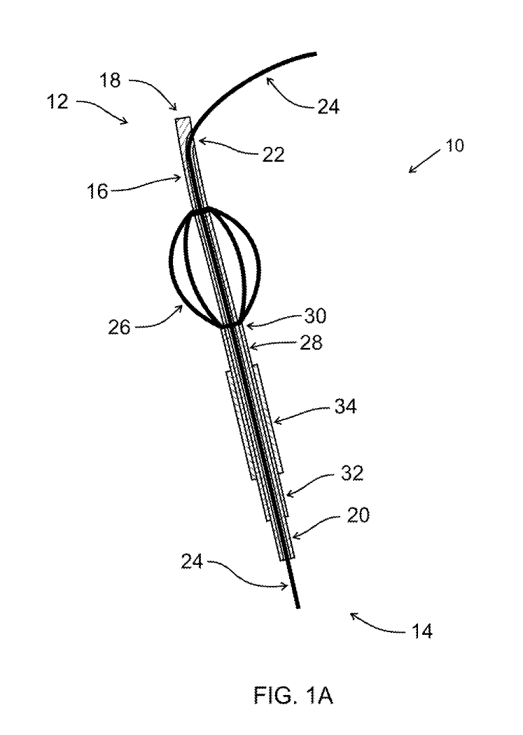

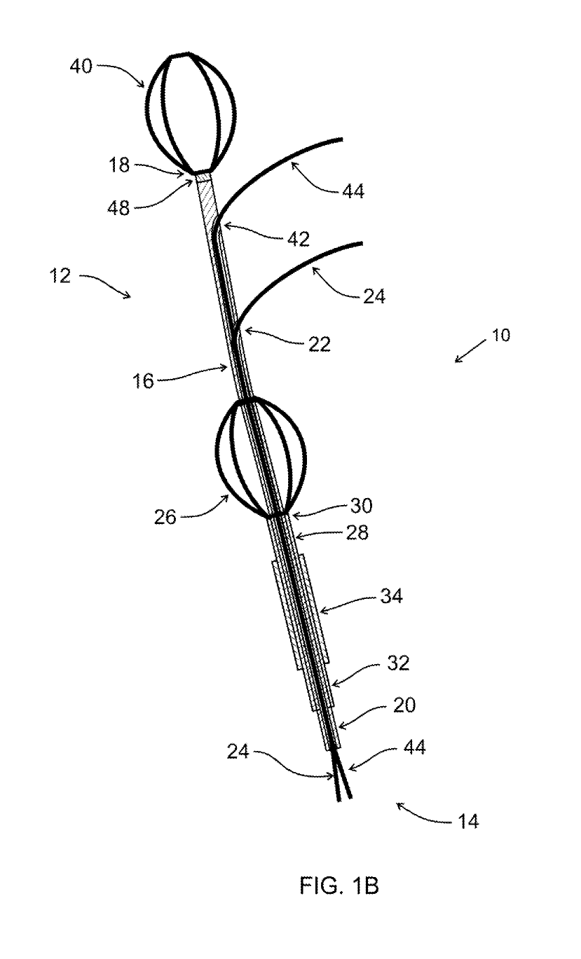

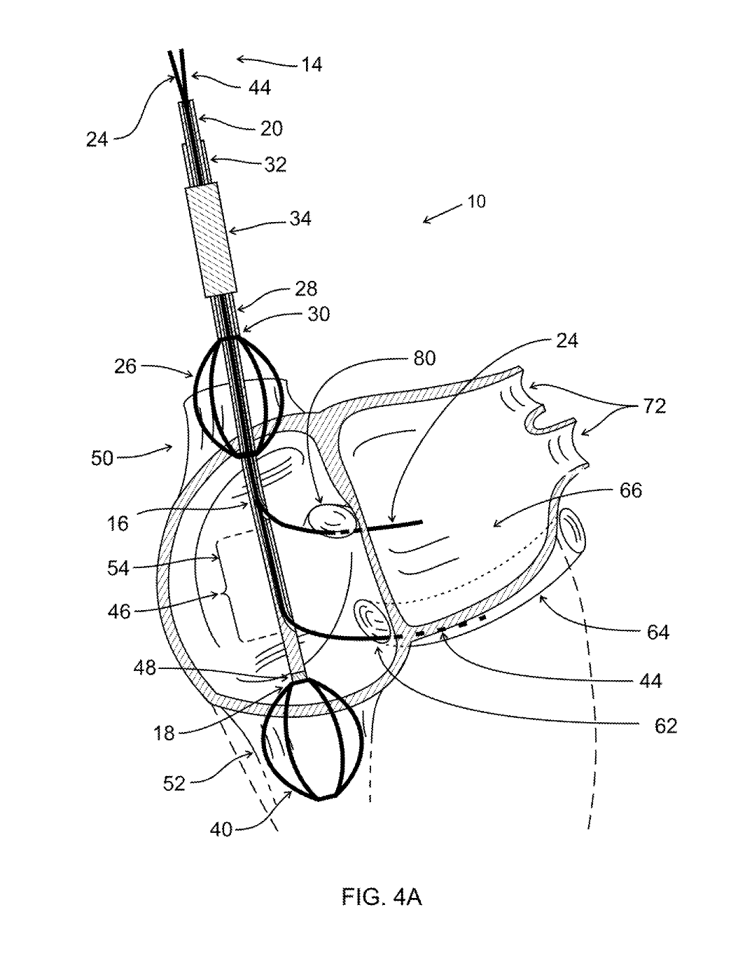

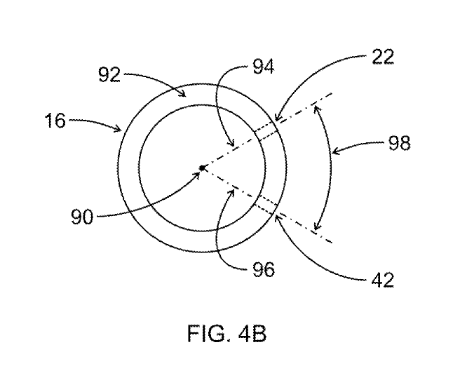

FIG. 1 illustrates a system for accessing a target within the body, for diagnosis or treatment. The figures and text herein illustrate and describe examples of the present invention for accessing structures of the heart or near the heart, but it is understood that the invention can be used for navigation and access in other areas of the body. For example, cardiac access system 10, while depicted and described and labeled as relating to navigating and accessing cardiac regions and structures, could be used in other areas of the body, and for accessing other target tissues and spaces, and be called, a “vascular access system”, “tissue access system”, “intestinal access system”, “neurological access system”, and so forth, with other tissues, structures, and elements named and described accordingly. Cardiac access system 10 comprises alignment catheter 16 with distal portion 12 to be located near the target, and proximal portion 14 having a conventional hub or manifold (not shown) for connection to other devices and/or for manipulation of various components and elements of cardiac access system 10. Alignment catheter 16 further comprises alignment catheter shaft 20 that extends between proximal portion 14 and distal portion 12. Cardiac access system 10 further comprises first stabilizing element 26 which can be deployed to stabilize distal portion 12 of alignment catheter 16 in a desired position within a body vessel or cavity, first directional element 24 which is directed away from alignment catheter axis 90 (FIG. 4B), and first alignment orifice 22 that directs first directional element 24 towards the target. Distal portion 12 of alignment catheter 16 is adapted for introduction to a body vessel, and can be advanced within the vasculature by conventional methods and tools, until distal portion 12 is located near the target. Alignment catheter 16 also has alignment catheter distal end 18.

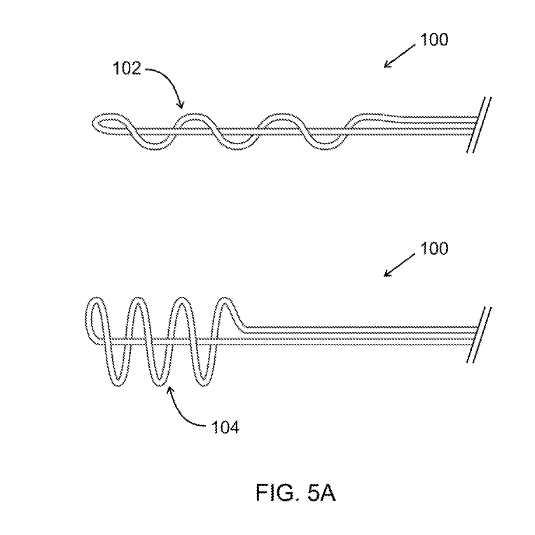

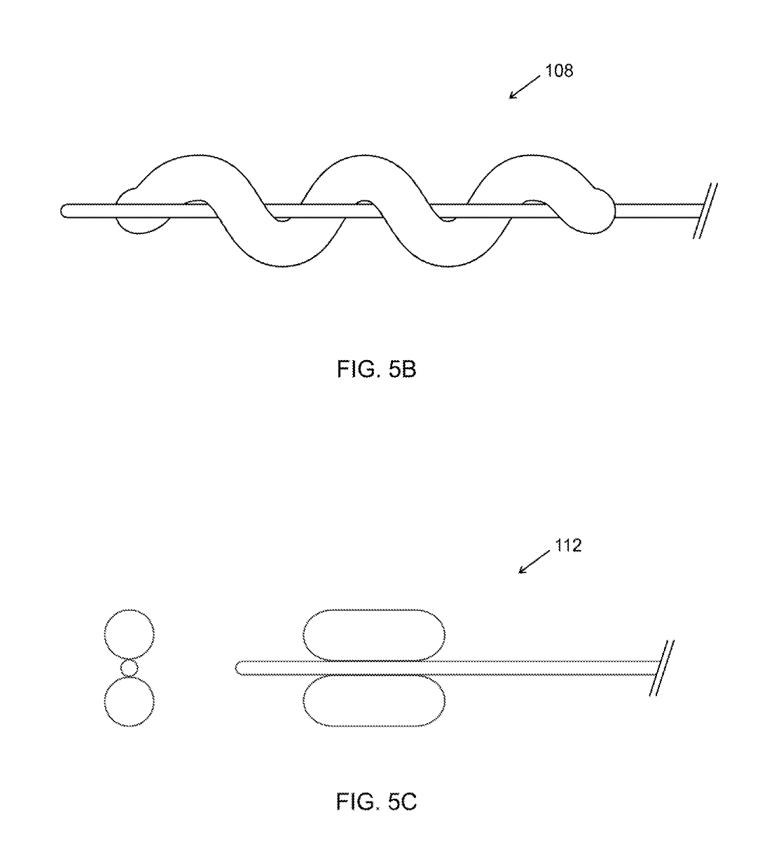

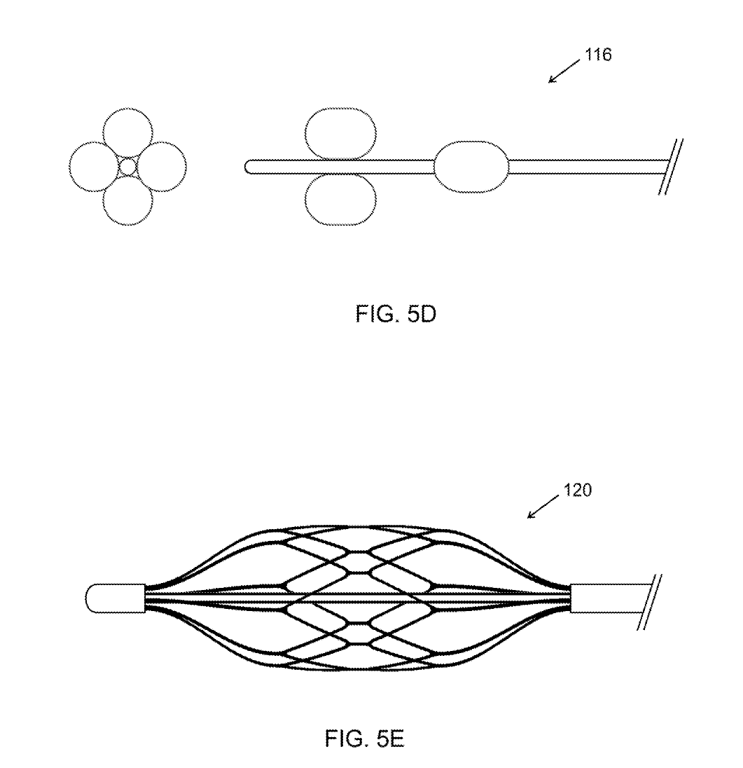

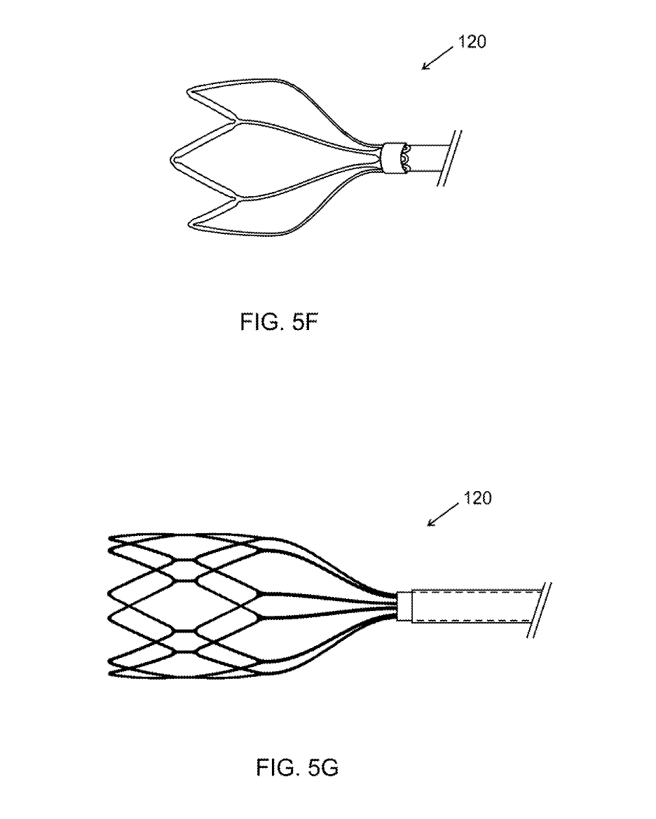

First stabilizing element 26 has a deployed configuration, in which the first stabilization element 26 is expanded or otherwise transformed to engage structures of the body vessel or chamber. For example, first stabilization element 26 can be a basket or cage that is deployed or expanded so that it engages the walls of the vessel, whereby the position and orientation of the cage is stabilized by the walls of the vessel. Alternatively, first stabilization element 26 can be a braid, or a stent, or one or more generally longitudinal struts that are placed in contact with the vessel wall when deployed. Still other alternatives are a ring structure, or a helical structure. Yet other alternatives are a balloon which is deployed by inflating the balloon until it contacts the vessel wall; the balloon can have a perfusion lumen to allow blood flow past the balloon, or can have a lobar cross sectional shape or other shape which does not completely occlude the vessel lumen when it is deployed. Various stabilizing means can be utilized for first stabilizing element 26; some examples of suitable stabilizing means are illustrated in FIG. 5A-5G. First stabilizing element 26 can be asymmetric to push alignment catheter shaft 20 into close approximation or direct contact with the wall of the vascular structure or other target. First stabilizing element 26 has a non-deployed configuration, in which first stabilizing element 26 is collapsed or otherwise transformed to a small profile for introduction into and passage within the vasculature or other tract in the body.

Cardiac access system 10 further comprises means for actuating first stabilizing element 26, comprising means for reversibly transforming first stabilizing element 26 between non-deployed and deployed configurations. For example, first stabilizing element 26 can be a self-expanding structure which is deployed by retraction of stabilizing element sheath 34, and non-deployed by advancement of the stabilizing element sheath 34 over the self-expanding structure. In another example, the first stabilizing element 26 can be a self-collapsing structure, which is deployed by bringing proximal and distal ends of the self-collapsing structure closer together, causing the central portion of the self-collapsing structure to expand; releasing the ends of the self-collapsing structure allows the self-collapsing structure to return to a non-deployed configuration. In yet another example, push-pull actuation is provided by sheath, catheter wire, or filament extending to the hub region of the catheter, whereby relative motions cause first stabilizing element 26 to transform between deployed and non-deployed configurations. In still another example, rotation of a control element causes first stabilizing element 26 to transform between deployed and non-deployed configurations. In a further example, fluid pressures or suctions are used to cause the stabilization element to transform between deployed and non-deployed configurations via fluid lumens along the catheter. Other alternatives include actuation by gears, linkages, electrical or electroactive actuators.

First directional element 24 is typically a catheter, sheath, guidewire, puncturing needle, or a combination. In some situations, the immediate goal is to place a guidewire or small catheter at the target, which is subsequently used to guide other devices to the target. In other situations, first directional element 24 comprises a puncturing needle which is used to pierce through tissue and is followed by other catheters, sheath, or devices. The location and orientation of first directional element 24 can be fixed, for example, by a fixed lumen of alignment catheter 16. In this case, distal portion 12 must be located and aligned within the body vessel or cavity to place first directional element 24 in the correct position; temporarily releasing or transforming first stabilizing element 26 to a non-deployed configuration, manipulating the access catheter position and orientation, and re-deploying first stabilizing element 26, can be performed multiple times as needed until the position and orientation of distal portion 12 places the first directional element 24 in the desired location and orientation to access the target. Even if the location and orientation of first directional element 24 are not quite perfect, once the location and orientation of first directional element 24 are close enough, then curved tip guidewires and catheters can be used as or with first directional element 24 to fine-tune the approach to properly access the target.

As an alternative to repeated repositioning of distal portion 12, controlled relative movement of first directional element 24 with respect to distal portion 12 can provided by alignment elements. Alignment elements can comprise a lumen in the access catheter, in which first directional element 24 can be moved axially and rotationally, so that the orientation of first directional element 24 can be adjusted by manipulation of proximal portion 14, to position and orient first directional element 24 as desired near distal portion, to properly access the target. Although the alignment catheter 16 may be repositioned to obtain a very general orientation at the distal portion, repeated repositioning to obtain careful positioning is not required, since the relative positioning of the directional element can be achieved by use of the alignment elements. First stabilizing element 26 can aid in this process by providing a stable location for distal portion 12. In some configurations, first stabilizing element 26 is affixed to alignment catheter 16. In other configurations, alignment catheter 16 is an inner catheter which can move axially and rotationally in a lumen of stabilizing element catheter 28, so that alignment catheter 16 can be used to orient and position first directional element 24 to properly access the target, while stabilizing element catheter 28 aids in controlling the position of first stabilizing element 26. In still other configurations, alignment elements comprise stabilizing element sheath 34 which engages the outer aspect of alignment catheter 16 so that stabilizing element sheath 34 can be manipulated to move alignment catheter 16 and first alignment orifice 22 so that first directional element 24 is manipulated axially and rotationally to properly access the target. Stabilizing element catheter 28 also comprises stabilizing element catheter shaft 32 and stabilizing element catheter distal end 30.

FIG. 1B illustrates another embodiment of cardiac access system 10, further comprising second stabilizing element 40, second alignment orifice 42 located an alignment distance 46 from first alignment orifice 22, and second directional element 44.

Note that in order to better show the invention apparatus and its functions and methods, portions of the human body are described and depicted in some of the figures; these elements are for reference only and in no way form part of the invented apparatus.

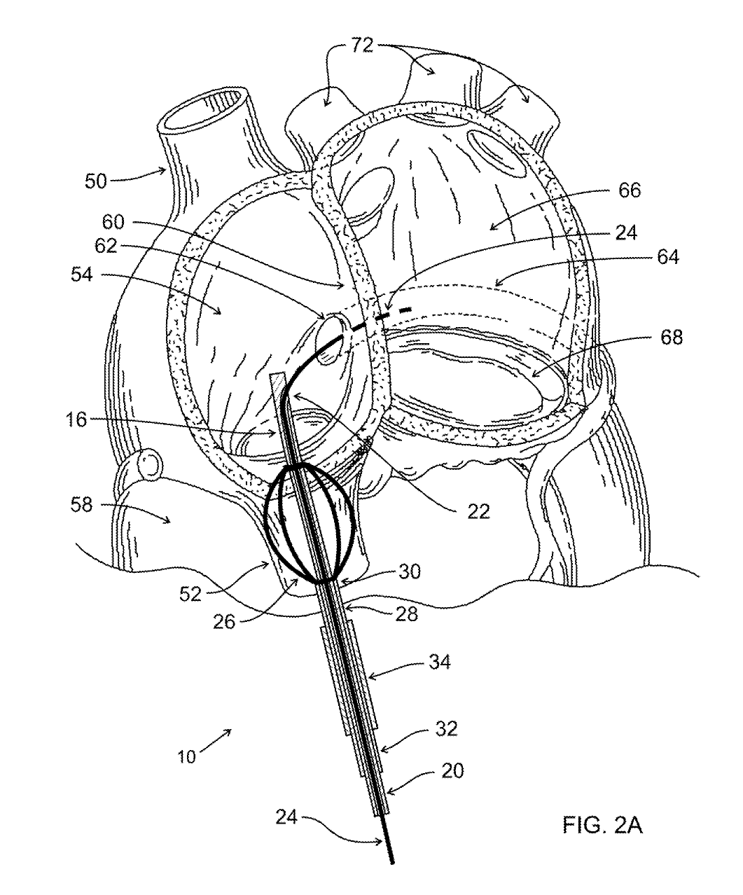

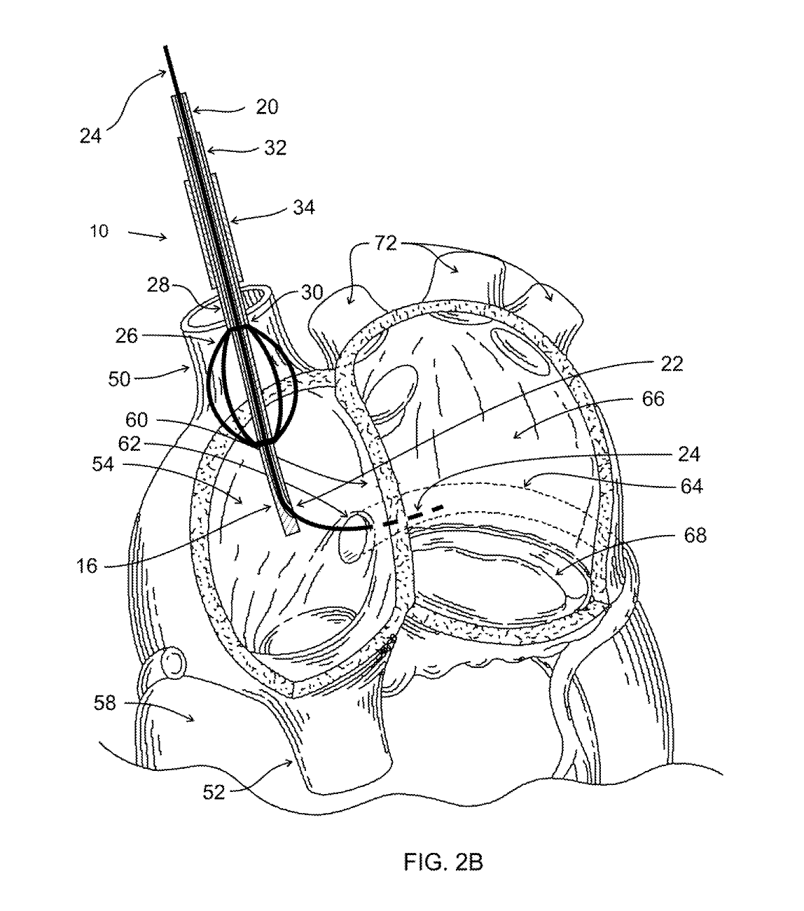

FIG. 2A illustrates the present invention applied to access the coronary sinus 64 from an inferior transvenous approach. In this example, elements of cardiac access system 10 such as that of FIG. 1A are introduced via percutaneous femoral vein access, and advanced cranially via the inferior vena cava 52 and positioned so that first stabilizing element 26 is secured at or near the right atrium 54; in this example, first stabilizing element 26 is deployed in the inferior vena cava 52, adjacent the right atrium 54. In this example, first stabilizing element 26 comprises a self-expanding basket, and means for deploying and un-deploying the basket comprises stabilizing element sheath 34 that is used to transform first stabilizing element 26 between a deployed (expanded) configuration and a non-deployed (collapsed) configuration. Stabilizing element sheath 34 is retracted, allowing first stabilizing element 26 to expand and contact the inferior vena cava 52, stabilizing distal portion 12. Alignment catheter 16 is used to direct first directional element 24 towards the coronary sinus ostium 62. In this example, alignment catheter 16 comprises an inner catheter within a lumen of stabilizing element catheter 28, with first directional element 24 directed by alignment catheter 16, which can be moved axially and rotationally within stabilizing element catheter 28 to locate and orient first directional element 24 for proper access to the coronary sinus ostium 62. In this example, first directional element 24 comprises a guidewire which is advanced from proximal portion 14 towards the coronary sinus ostium 62. After passing through the coronary sinus ostium 62, first directional element 24 (in this example, a guidewire) can be advanced into the coronary sinus 64 as needed. The orientation of first directional element 24 can be adjusted, such as by rotating or otherwise manipulating elements near distal portion 12 without contracting first stabilizing element 26, or by temporarily contracting first stabilizing element 26 and rotating stabilizing element catheter 28 as needed, according to imaging or other guidance. Once the orientation of first directional element 24 is as desired, first directional element 24 is advanced in the direction desired, for example, to access the coronary sinus ostium 62. In the case that first directional element 24 comprises a directional catheter, and the tip of the directional catheter is directed into, at, or near the coronary sinus ostium 62, the coronary sinus ostium 62 and the coronary sinus 64 is accessed by further advancement of the directional catheter, or by advancing a guidewire out from the distal portion of the directional catheter.

FIG. 2B illustrates the present invention applied to access the coronary sinus 64 from a superior transvenous approach. The elements and methods are similar to those of FIG. 2A, but adapted for a superior approach. In this case, a subclavian vein or a jugular vein, for example, can be used for access. In some situations, and for some target tissues and locations, a superior approach is preferred. For example, the coronary sinus 64 is often more readily accessed from a superior approach due to the presence of valve tissue adjacent the coronary sinus ostium 62 that can make entry from an inferior approach difficult. Also, a superior access across the interatrial septum 60 from the right atrium 54 may be preferred to provide a more direct alignment with the axis of the mitral valve 68.

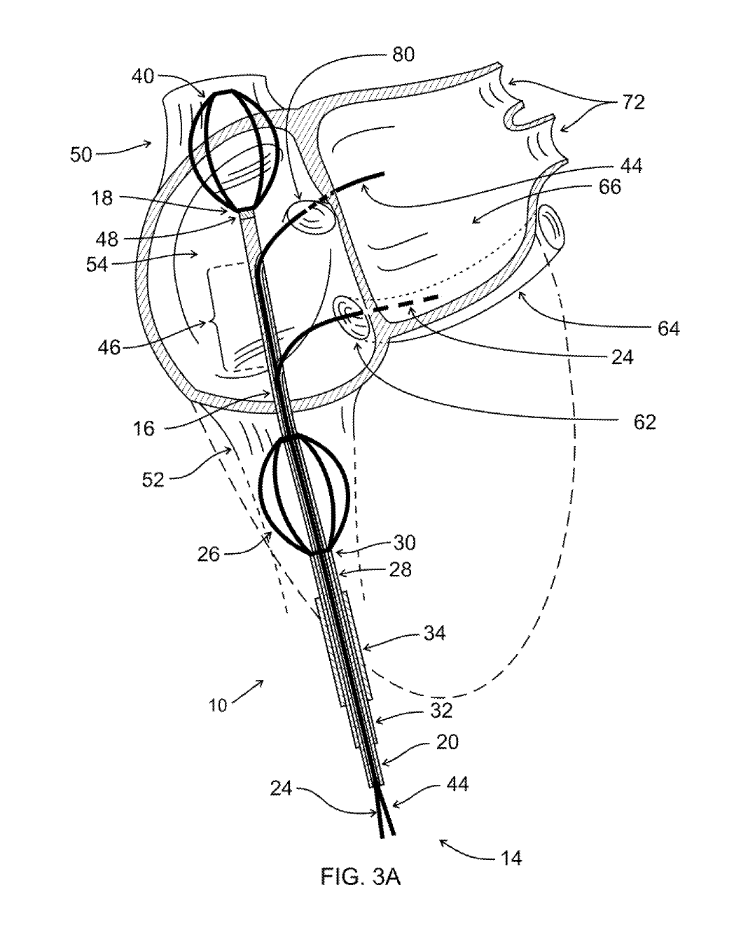

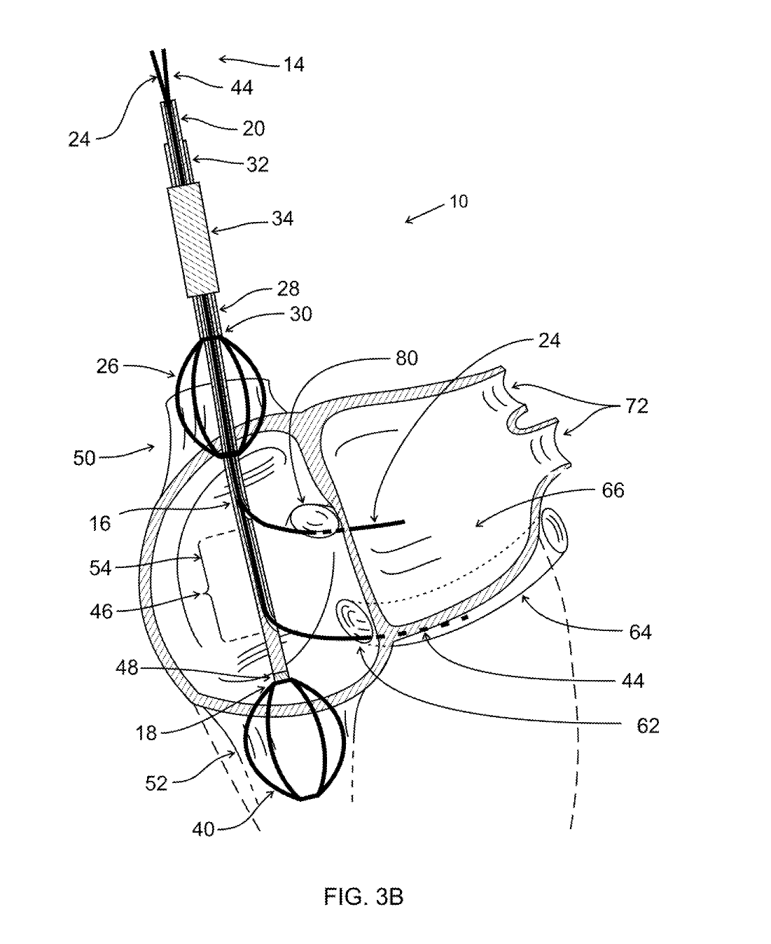

FIG. 3A illustrates other embodiments of the present invention, in which cardiac access system 10 comprises an additional stabilization element, and an additional directional element, such as that of FIG. 1B. Note that cardiac access system 10 can comprise one or more stabilizing elements, with one or more directional elements, in any combination, as long as there is at least one directional element and at least one other directional and/or stabilizing elements; (i.e., two or more directional elements, or at least one directional element and at least one stabilizing element). In the example illustrated in FIG. 3A, cardiac access system 10 comprises two stabilizing elements, first stabilizing element 26 positioned in or near the inferior vena cava 52 or within the right atrium 54, and second stabilizing element 40 positioned in the superior vena cava 50 or within the right atrium 54, with two directional elements (first directional element 24 and second directional element 44) disposed between the two stabilizing elements. This configuration provides additional stabilization of distal portion 12 of the access catheter, without depending on a particular geometry of the right atrium 54. First stabilizing element 26 and second stabilizing element 40, which in this example are illustrated as baskets, can alternatively be various stabilizing means such as those illustrated in FIGS. 5A-5G.

In one method, the apparatus is used as illustrated in FIG. 3A by introducing portions of cardiac access system 10 into the body and advancing distal portion 12 to a position in or near the right atrium 54, then first directing first directional element 24 (such as a guidewire or small catheter) into the coronary sinus 64, similar to the approach previously described with respect to FIG. 2A, except that two stabilizing elements are deployed. After confirming that first directional element 24 is correctly positioned in the coronary sinus 64, second directional element 44 is directed to a desired location such as the fossa ovalis 80, or other location desired for trans-septal access. The known locational relationship between the coronary sinus ostium 62 and the fossa ovalis 80, for example, is used to orient second directional element 44 properly, either being fixed by orientation (such as alignment angle offset 98, FIG. 4B) and locations (such as alignment distance 46, FIG. 3A) of lumens and orifices (such as first alignment orifice 22 and second alignment orifice 42, FIG. 3A) of alignment catheter 16, or by being adjustable by the user. In other methods, access from a superior location, such as illustrated in FIGS. 2B and 3B, can be used, but with two directional elements, and optionally, at least one stabilizing element.

In yet other embodiments, the apparatus includes at least two stabilizing elements, but only a single directional element. The at least two stabilizing elements can be jointly deployed, using a single actuation mechanism, or can be deployed independently.

The target can be a specific tissue, or a particular location within the body, or a location along the way where specific guidance is required in order to reach a specific tissue or location. For example, the target can be the myocardial muscle sleeve around a pulmonary vein ostium, which is where pulmonary veins 72 drain into left atrium 66, for placement of electrophysiology device at that location. In another example, the target can be the blood-filled space within the left atrial appendage, to evaluate stasis. In yet another example, the target can be a location in the interatrial septum which is desired to puncture in order to gain access to place a device at the mitral valve.

Various directional and/or distance and/or other orientation indicators as are known in the art can be incorporated, such as radiopaque markings, markings along alignment catheter shaft 20, indicators at proximal portion 14, and so forth.

Various adjustment features can be incorporated to provide controlled movement of the directional element(s) with respect to the stabilizing element(s), to provide for directing the directional element(s) at a desired alignment distance and alignment angle(s) orientation with respect to the stabilizing element(s) and/or the directional elements(s).

When a percutaneous approach is not chosen, such as because of the size of a therapy device which must be placed, or other difficulty, tissues and locations in and near the heart can be accessed using “keyhole”, “mini-thoracotomy”, apical access, and other surgical approaches and hybrid approaches, which can still benefit from the present invention. For example, small, elongated tools and/or catheters can be placed through a small incision, and the present invention can be used to access and guide various devices to the particular tissue or location in the body. In other cases, a strictly percutaneous approach may not be feasible due to anatomical constraints, but a small surgical cut-down can be used to approach a blood vessel that is used for access, with catheter-based devices than navigating through the vascular system; the present invention can be useful in these cases as well, to facilitate safe and reliable access to the desired target tissue or location. The initial access site to a vessel may be remote, or may be relatively close to the target tissue or location. Central, or peripheral, close, or distant, access is anticipated, with the present invention providing access to the target tissue or location.

While navigating and directing a device within a body vessel or chamber such as a blood vessel or chamber of the heart is a particularly advantageous application in which the present invention finds utility, other uses are anticipated, including navigating within the urinary system, or a cerebrospinal fluid space, or within the trachea or bronchial tree, or within the gastrointestinal system, or extravascular access path, which can also benefit from the stabilization and directional control obtained with the present invention, as can a variety of less-invasive or minimally-invasive surgical approaches such as keyhole surgery, laparoscopic procedures, endoscopic procedures, and so forth.

FIGS. 4A and 4B show an alternate embodiment of the present invention. Cardiac access system 10 comprises an outer catheter such as stabilizing element catheter 28, comprising first stabilizing element 26 located near or at the distal end of stabilizing element catheter 28. In an alternate embodiment first stabilizing element 26 is omitted from the outer catheter. The outer catheter can, for example, be an introducer sheath placed into a vein such as the subclavian vein at the access site. First stabilizing element 26, of the embodiment of FIG. 4A can be an expandable basket or coil (such as illustrated in FIGS. 1A-4A, 5A, 5E-5G) or balloon(s) (such as those of FIGS. 5B-5D) as described herein. The stabilizing element(s) can be symmetrical about the axis of the outer catheter, or can be asymmetric to hold stabilizing element catheter shaft 32 proximate the interatrial septum 60.

Located within the outer catheter and extending through a manifold located at the proximal end of the outer catheter and extending out the distal end of the outer catheter is alignment catheter. Alignment catheter 16 has a first alignment orifice located in the distal portion of alignment catheter 16 which comprises first alignment orifice 22 and second alignment orifice 42. First alignment orifice 22 can be located in alignment catheter side wall 92 of alignment catheter 16 to orient first alignment orifice 22 at a first alignment angle 94 with respect to the alignment catheter axis 90. Alternatively, first alignment orifice 22 can be located at the distal end of alignment catheter 16.

Located in alignment catheter side wall 92 of alignment catheter 16 is second alignment orifice 42. Second alignment orifice 42 is located at alignment distance 46 along alignment catheter shaft 20 in a proximal direction toward the catheter manifold. Second alignment orifice 42 is also oriented at second alignment angle 96 with respect to the first alignment orifice. First alignment angle 94 can be the same as second alignment angle 96, or can there can be an alignment angle offset 98 between them.

First directional element 24 (such as a catheter or guidewire) extends along alignment catheter 16 and extends through the manifold at the proximal portion 14 of alignment catheter 16 and extends out through first alignment orifice 22. In one embodiment alignment catheter 16 directs first directional element 24 catheter through first alignment orifice 22 through alignment catheter side wall 92 of alignment catheter 16; first directional element 24 being directed at first alignment angle 94 with respect to the axis of the alignment catheter. In another embodiment, first directional element 24 extends through the distal end of alignment catheter 16; in this embodiment first directional element 24 is a catheter is formed with a curved shape that directs first directional element at a first alignment angle 94 with respect to the axis of alignment catheter 16; in this embodiment, the shaft of first directional element 24 is keyed at the location of the manifold or elsewhere along its length such that first alignment angle 94 is known and fixed with respect to the axis of alignment catheter 16.

Second directional element 44 (such as a catheter or guidewire) extends within alignment catheter 16 extending through the manifold of alignment catheter 16 and extending out through second alignment orifice 42. Alignment catheter 16 directs second directional element 44 through second alignment orifice 42 at second alignment angle 96 with respect to the axis of alignment catheter 16. First directional element 24 and second directional element 44 are thereby directed with alignment angle offset 98 relative to each other with respect to the axis of alignment catheter 16.

First directional element 24 can comprise a guidewire, a sheath or catheter that extends over the guidewire, or both. A guidewire, for example, can first be directed into a first target such as the coronary sinus ostium 62. A sheath or catheter can then follow over the guidewire and into the coronary sinus ostium 62. Contrast can be delivered through the sheath or catheter to verify location within the coronary sinus 64 using radiographic, MRI, ultrasound, or other imaging.

Second directional element 44 can similarly comprise a guidewire, a sheath, or catheter. In one example, second directional element 44 comprises a curved hollow needle with an external protective sheath for protection against accidental puncture or other tissue injury; the protective sheath can be withdrawn to expose the needle tip for puncture across the interatrial septum. Second directional element 44 can further comprise a guidewire contained within the needle, for advancement into the left atrium 66 once access had been made across the interatrial septum 60.

A method of use, for example, can be as follows. This example provides a safe and reliable access to cross the interatrial septum by first locating the coronary sinus ostium 62 and using the coronary sinus 64 access to facilitate entry across the interatrial septum 60 at a desired location. It is understood that other locations and orifices, such as the tricuspid valve (not shown), which is situated between right atrium 54 and right ventricle 58, or other suitable locations, can be used similarly to facilitate access across the interatrial septum 60. It is also understood that the apparatus can be used to gain access to other orifices, chambers, and tissues in the body.

Access to the left atrium via the superior approach via the superior vena cava offers several advantages. First, access to the coronary sinus is most easily accomplished via a superior approach. This is in part due to the valve located adjacent the coronary sinus ostium which is difficult to access from the inferior vena cava. Second, a superior approach to cross the interatrial septum is made easy and reliable with the use of this apparatus. Normally access across the septum is gained from an inferior position using the inferior vena cava and providing access across the septum via the fossa ovalis or a patent foramen ovale. This access from an inferior position however can require that a catheter that crosses the septum form a loop within the left atrium, especially if one is attempting to gain access into the mitral valve. For mitral valve replacement or mitral repair, the catheters are often large and stiff and are unable to properly align with the axis of the mitral annulus after forming such a loop. This is overcome by with the present invention by facilitating superior access via the superior vena cava. Location of the desired trans-septal puncture location of the interatrial septum from a superior position, however, has heretofore been not reliable or safe using current delivery catheters due to the possibility of accidently puncturing the aorta, esophagus, or other adjacent structures; these difficulties are overcome by the present invention.

By first obtaining access to one location that is easily accessed from above, such as the coronary sinus ostium, the present catheter then provides an appropriate distance and angle to directly access a second location such as the interatrial septum safely. For example, the coronary sinus is located between the left ventricle and the left atrium and drains into the right atrium. The fossa ovalis in the interatrial septum is typically located superior to the coronary sinus ostium and in line with the superior vena cava. Therefore, the present apparatus allows the distance and angle from the coronary sinus ostium to be set to locate a safe and reliable puncture across the septum. Access across the septum from a superior position allows a more direct alignment with the mitral valve axis and therefore allows a large and stiff catheter to be delivered to the mitral annulus with improved alignment for performing transcatheter mitral valve replacement or repair.

In one example, an outer catheter contained within an external sheath is placed via the subclavian vein through an introducer sheath and over a guidewire (and also with a nosecone or tapered leading element, not shown, to follow closely over the guidewire) to the right atrium. The outer sheath is retracted to allow a self-expanding basket to enlarge in diameter and make contact with the superior vena cava or right atrium to hold the apparatus firmly in place. An inner alignment catheter is advanced to place the first alignment orifice near the coronary sinus ostium and a first directional catheter or guidewire is advanced into the coronary sinus. A contrast injection can be used to confirm proper positioning in the coronary sinus. A second directional catheter such as a hollow puncture needle is advanced within the alignment catheter and is directed at the interatrial septum at a known location relative to the first directional catheter. Advancement of puncture means such as a hollow needle at a known location provides safe and reliable access across the septum; a guidewire is then placed across the septum through the puncture hole made by the needle. The guidewire is used to provide access across the interatrial septum to further devices, such as to perform mitral replacement or repair, to close the left atrial appendage (not shown), to ablate atrial fibrillation foci, or other diagnostic or therapeutic procedure. The outer sheath can be used to provide a conduit for delivery of the mitral catheter if desired.

FIG. 5A illustrates stabilizing means comprising expandable coil stabilizing element 100, illustrating expandable oil 102, and also showing expandable coil expanded 104.

FIG. 5B illustrates stabilizing means comprising helical balloon stabilizing element 108 which can be expanded by inflation to provide stabilization while allowing blood flow past the balloon. Stabilizing element 108 is illustrated in the expanded configuration.

FIG. 5C illustrates stabilizing means comprising dual opposed balloons stabilizing element 112, which similarly can be expanded by inflation to provide stabilization while allowing blood flow past the balloons. Stabilizing element 112 is illustrated in the expanded configuration.

FIG. 5D illustrates stabilizing means comprising staggered opposed balloons stabilizing element 116, which similarly can be expanded by inflation to provide stabilization while allowing blood flow past the balloons. Stabilizing element 116 is illustrated in the expanded configuration.

FIG. 5E illustrates stabilizing means comprising dual end constrained expandable basket stabilizing element 120 which can be expanded by push-pull actuation, for example, to provide stabilization while allowing blood flow past the basket. Stabilizing element 120 is illustrated in the expanded configuration.

FIG. 5F illustrates stabilizing means comprising single end constrained expandable basket stabilizing element 124 which can be expanded by self expansion when advanced from an external sheath, for example, to provide stabilization while allowing blood flow past the basket. Stabilizing element 124 is illustrated in the expanded configuration.

FIG. 5G illustrates stabilizing means comprising an alternative single end constrained expandable basket stabilizing element 128 which can be expanded by self expansion when advanced from an external sheath, for example, to provide stabilization while allowing blood flow past the basket. Stabilizing element 128 is illustrated in the expanded configuration.

Various other stabilizing means such as rings, perfusion balloons, spokes, and combinations of the disclosed elements can be utilized.

In some embodiments, the present invention is a cardiac access catheter comprising a first alignment orifice, and a stabilizing element, as described herein. In other embodiments, the present invention is a cardiac access catheter comprising a first alignment orifice, and a second alignment orifice, as described herein. In yet other embodiments, the present invention is a cardiac access catheter comprising a first alignment orifice, a second alignment orifice, and a stabilizing element, as described herein. In yet other embodiments, the present invention is a catheter system, comprising at least one catheter and with various elements as described herein distributed between multiple components, catheters, sheaths, guidewires, needles, dilators, and so forth. In still other embodiments, the present invention is a process for accessing target tissues and locations within the body, as described herein. In further embodiments, the present invention is a method of using catheters and systems as described herein, to access target tissues and locations within the body, to perform a diagnostic or therapeutic procedure.

Various modifications can be made to the present invention without departing from the apparent scope thereof.

It is claimed:

1. A catheter system for transvascular access to target locations in the body, comprising:
an alignment catheter having a distal portion and a first alignment orifice;
a stabilizing element catheter having at least one stabilization element located near said distal portion, said at least one stabilization element having a collapsed introduction configuration and an expanded deployed configuration which places portion(s) of said at least one stabilization element in contact with a body vessel or tissue near the target location while allowing fluid flow past said stabilization element;
means for reversibly actuating said at least one stabilization element between the collapsed introduction configuration and the expanded deployed configuration;
said stabilizing catheter having a lumen for passage of said alignment catheter,
a first directional element;
said alignment catheter having a lumen which communicates with said first alignment orifice and provides for passage of said first directional element; and,
wherein said first directional element is extendable through said first alignment orifice to be thereby directed towards a target location.

2. The catheter system of claim 1, wherein:
said means for reversibly actuating said at least one stabilization element between the collapsed introduction configuration and the expanded deployed configuration comprises a stabilizing element sheath.

3. The catheter system of claim 1, wherein said at least one stabilization element comprises at least two stabilization elements.

4. The catheter system of claim 1, wherein said first directional element comprises a needle for trans-septal puncture.

5. The catheter system of claim 1, wherein said first directional element comprises a guidewire.

6. The catheter system of claim 1, wherein said first directional element comprises a catheter with a lumen.

7. A catheter system for transvascular access to target locations in the body, comprising:
an alignment catheter having a distal portion and a first alignment orifice;
a first directional element
a second directional element;
said alignment catheter having a lumen which communicates with said first alignment orifice and provides for passage of said first directional element;
wherein said first directional element is extendable through said first alignment orifice to be thereby directed towards a target location;
a second alignment orifice communicating with a lumen for passage of said second directional element; and,
wherein said second directional element is extendable through said second alignment orifice to be thereby directed towards a target location.

8. The catheter system of claim 7, wherein:
the location of said second alignment orifice relative to the location of said first alignment orifice is adjustable.

9. The catheter system of claim 7, wherein said first directional element comprises a catheter with a lumen.

10. The catheter system of claim 7, wherein said first directional element comprises a guidewire.

11. The catheter system of claim 7, further comprising:
a stabilizing element catheter having at least one stabilization element located near said distal portion, said at least one stabilization element having a collapsed introduction configuration and an expanded deployed configuration which places portion(s) of said at least one stabilization element in contact with a body vessel or tissue near the target location while allowing fluid flow past said stabilization element;
means for reversibly actuating said at least one stabilization element between the collapsed introduction configuration and the expanded deployed configuration; and,
said stabilizing catheter having a lumen for passage of said alignment catheter.

12. A method for accessing a target location in the body, comprising the steps of:
providing an alignment catheter with a distal portion, a first alignment orifice, a second alignment orifice, a first directional element, and a second directional element;
introducing the alignment catheter into the body and advancing the alignment catheter to a location near the target location;
advancing the first directional element through the first alignment orifice into a body lumen near the target location;
using the first directional element in the body lumen near the target location to locate the second alignment orifice in a position to direct the second directional element; and,
advancing the second directional element to the target location.

13. The method of claim 12 further comprising the step of adjusting the relative position of the second alignment orifice with respect to the location of the first alignment orifice.

14. The method of claim 12 further comprising the steps of:
providing a stabilization element and means of actuating the stabilization element;
using the stabilization element to stabilize the distal portion of the alignment catheter while advancing the second directional element to the target location.

15. The method of claim 12 wherein the body location near the target location is the coronary sinus, and further comprising the steps of:
providing a puncture device; and,
using the puncture device to puncture the interatrial septum to access the left atrium.

16. The method of claim 15 wherein the step of introducing the alignment catheter into the body and advancing the alignment catheter to a location near the target location is done from a superior venous access passing caudally through the superior vena cava.
